(19) United States
(12) Patent Application Publication
(10) Pub. No.: US 2020/0222072 A1
(43) Pub. Date: Jul. 16, 2020

(54) IMAGE-GUIDED PHARYNGOLARYNGEAL FORCEPS ASSEMBLY

(71) Applicant: Juen-Haur HWANG, Taichung City (TW)

(72) Inventor: Juen-Haur HWANG, Taichung City (TW)

(21) Appl. No.: 16/244,577

(22) Filed: Jan. 10, 2019

(51) Int. Cl. A61B 17/29; A61B 17/24; A61B 1/267

(52) U.S. Cl. CPC A61B 17/29; A61B 17/24; A61B 1/05; A61B 2017/2904; A61B 2017/2905; A61B 1/267

(57) ABSTRACT

An image-guided pharyngolaryngeal forceps assembly includes a pair of pharyngolaryngeal forceps and an image-guided device. The image-guided device is fixed on a body of the pair of pharyngolaryngeal forceps, is slender and flexible, and has a tube body, a camera module and a signal transmitter. The tube body is mounted along the body of the pharyngolaryngeal forceps. The camera module is connected to an end of the tube body near a jaw assembly of the pair of pharyngolaryngeal forceps, and has a lens and multiple LEDs. The signal transmitter, such as a connector or a wireless signal transmitter, is connected to an end of the tube body near a handle of the pair of pharyngolaryngeal forceps, and can transmit signals to an external device. The image-guided pharyngolaryngeal forceps assembly can capture and transmit real time images to an external device and can provide illumination during a pharyngolaryngeal surgery.

### BACKGROUND OF THE INVENTION

### 1. Field of the Invention

The present invention relates to a surgical instrument for pharynx and larynx, and more particularly to an image-guided pharyngolaryngeal forceps assembly.

### 2. Description of Related Art

A pair of conventional pharyngolaryngeal forceps 90 for a pharyngolaryngeal foreign body removal surgery, as shown in FIG. 6, has a jaw assembly 91, a slender and curved body 93 for easy insertion into the pharyngolaryngeal cavity, and a handle 95 applied to control opening and closing movements of the jaw assembly 91.

To operate a pharyngolaryngeal foreign body removal surgery in an outpatient clinic, the patient needs to sit up straight and hold the tongue out of the mouth to unblock the opening of the pharyngolaryngeal cavity. The surgeon needs to wear a light source on the head, hold an indirect pharyngolaryngeal mirror 80 with one hand, and hold laryngeal forceps 90 with the other hand. Then, the surgeon will insert the body 93 of the pharyngolaryngeal forceps 90 into the pharyngolaryngeal cavity of the patient, and will grasp and remove foreign bodies from the pharynx or larynx with the jaw assembly 91 under the reflective field of view of the indirect pharyngolaryngeal mirror 80.

However, the indirect pharyngolaryngoscopy surgery has the following shortcomings. The patient needs to hold the tongue by his/her own hand to unblock the opening of laryngeal cavity, and may not cooperate well with the surgeon. Moreover, the reflective field of view of the indirect pharyngolaryngeal mirror 80 is small. The surgeon may not see foreign bodies in the pharynx or larynx clearly under insufficient light with the indirect pharyngolaryngeal mirror 80, and this will cause failure of foreign body removal.

To overcome the shortcomings, the present invention tends to provide an image-guided pharyngolaryngeal forceps assembly to mitigate or obviate the aforementioned problems.

### SUMMARY OF THE INVENTION

The main objective of the invention is to provide a simple pharyngolaryngeal surgical instrument, which can provide illumination and capture real time videos or images of pharynx and larynx during a pharyngolaryngeal surgery. The surgeon can perform the surgery without holding an indirect laryngeal mirror and wearing a lighting source for observing assistance.

The image-guided pharyngolaryngeal forceps assembly has a pair of pharyngolaryngeal forceps and an image-guided device. The pair of pharyngolaryngeal forceps has a jaw assembly, a body connected to the jaw assembly and being slender and curved, and a handle connected to the jaw assembly via the body. The image-guided device is fixed on the body of the pharyngolaryngeal forceps and has a tube body, a camera module, and a signal transmitter. The tube body is mounted along the body of the pharyngolaryngeal forceps, is slender and flexible, and has multiple electrical wires mounted in the tube body. The camera module is disposed on an end of the tube body near the jaw assembly of the pharyngolaryngeal forceps and has a lens and multiple light-emitting diodes (LEDs) facing the jaw assembly of the pharyngolaryngeal forceps. The signal transmitter is disposed on an end of the flexible tube body near the handle of the pharyngolaryngeal forceps and is connected to the camera module with the electrical wires of the tube body.

Other objects, advantages and novel features of the invention will become more apparent from the following detailed description when taken in conjunction with the accompanying drawings.

### BRIEF DESCRIPTION OF THE DRAWINGS

FIG. 1 is a side view of a first embodiment of an image-guided pharyngolaryngeal forceps assembly in accordance with the present invention;

FIG. 2 is an enlarged perspective view of an image-guided device of the image-guided pharyngolaryngeal forceps assembly in FIG. 1;

FIG. 3 is an operational side view of the image-guided pharyngolaryngeal forceps assembly in FIG. 1;

FIG. 4 is an operational side view of a second embodiment of an image-guided pharyngolaryngeal forceps assembly in accordance with the present invention;

FIG. 5 is an enlarged perspective view of the image-guided pharyngolaryngeal forceps assembly in FIG. 1 showing the image-guided device fixed on the body of the pharyngolaryngeal forceps with wire holders;

FIG. 5A is an enlarged perspective view of the image-guided pharyngolaryngeal forceps assembly in accordance with the present invention showing the image-guided device fixed in a holder portion of the body of the pharyngolaryngeal forceps; and

FIG. 6 is an operational side view of conventional pharyngolaryngeal forceps.

### DETAILED DESCRIPTION OF PREFERRED EMBODIMENT

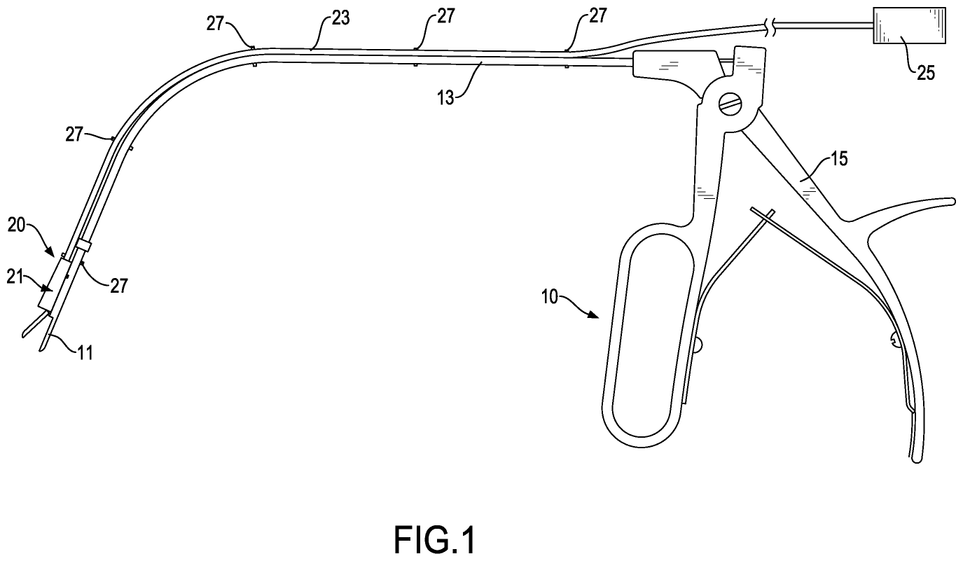

With reference to FIG. 1, an image-guided pharyngolaryngeal forceps assembly in accordance with the present invention has a pair of pharyngolaryngeal forceps 10 and an image-guided device 20.

The pair of pharyngolaryngeal forceps 10 may be conventional, is applied to grasp foreign bodies in the pharynx or larynx or remove diseased tissues of the pharynx or larynx of a patient, and has a jaw assembly 11, a body 13, and a handle 15. The jaw assembly 11 is applied to grasp foreign bodies in or diseased tissues of the pharynx or larynx. The jaw assembly 11 may have various specifications for different pharyngolaryngeal surgeries, and the detail thereof is omitted. The body 13 is connected to the jaw assembly 11, and is slender and curved for easy insertion into the pharyngolaryngeal cavity. The handle 15 may be formed as a scissors handle, is connected to the jaw assembly 11 via the body 13, and is applied to control opening and closing movements of the jaw assembly 11 to grasp and hold foreign bodies and diseased tissues.

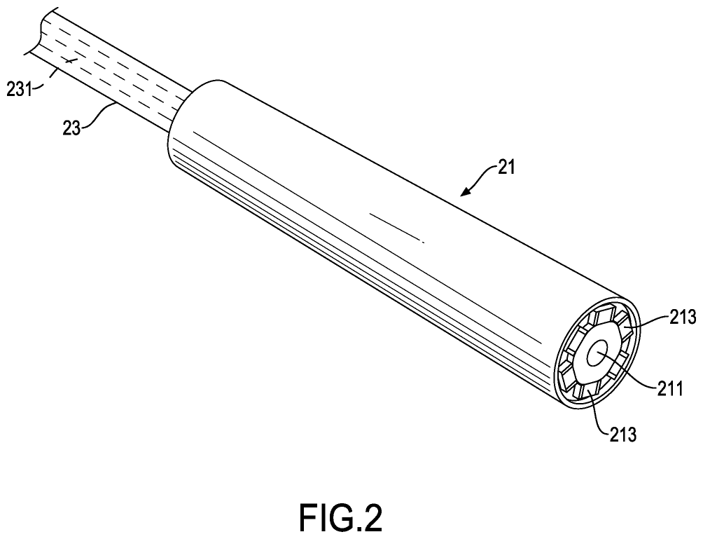

With reference to FIGS. 1 and 2, the image-guided device 20 is flexible, slender, and tubular, and is fixed on the body 13 of the pharyngolaryngeal forceps 10 along the body 13 of the pharyngolaryngeal forceps 10. The image-guided device 20 has a tube body 23, a camera module 21, and a signal transmitter 25. The tube body 23 is mounted along the body 13 of the pharyngolaryngeal forceps 10, is flexible and slender, and has electrical wires 231 mounted in the tube body 23. The camera module 21 and the signal transmitter 25 are respectively disposed on opposite ends of the tube body 23 and are connected to each other with the electrical wires 231. The camera module 21 is disposed on one of the ends of the tube body 23 near the jaw assembly 11, and the signal transmitter 25 is disposed on the other end of the tube body 23 near the handle 15.

The camera module 21 is a cylinder and has a lens 211 and multiple light-emitting diodes (LEDs) 213 mounted in the end of the camera module 21 that faces the jaw assembly 11 of the pharyngolaryngeal forceps 10. The lens 211 is mounted in the middle of the camera module 21 to capture videos or images near the jaw assembly 11. The LEDs 213 are arranged around the lens 211 at angular intervals to illuminate the area where the lens 211 is capturing images.

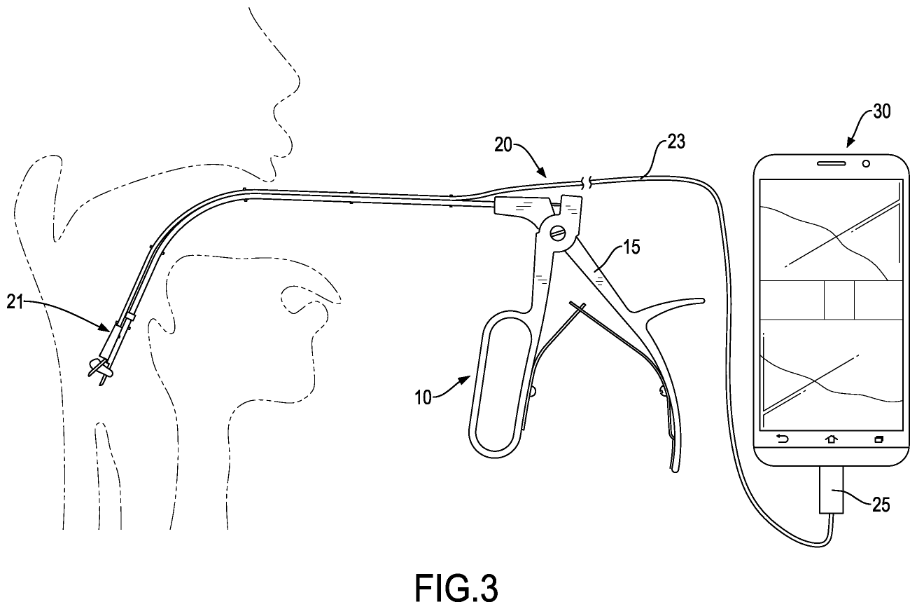

With reference to FIG. 3, in the first embodiment, the signal transmitter 25 is an electrical connector, such as a universal serial bus connector (e.g., USB2.0, USB3.0, USB Type-C, mini USB, or Micro USB connector) or any other connector, which can be connected to an external device 30 (e.g., smart phone, tablet computer, or personal computer) to transmit information and supply power to the image-guided device 20. After the electrical connector is connected to the external device 30, the external device 30 can supply power to the camera module 21 via the electrical connector, the image-guided device 20 can transmit signals to the external device 30, and the real time video or image being captured by the camera module 21 can be displayed on the screen of the external device 30.

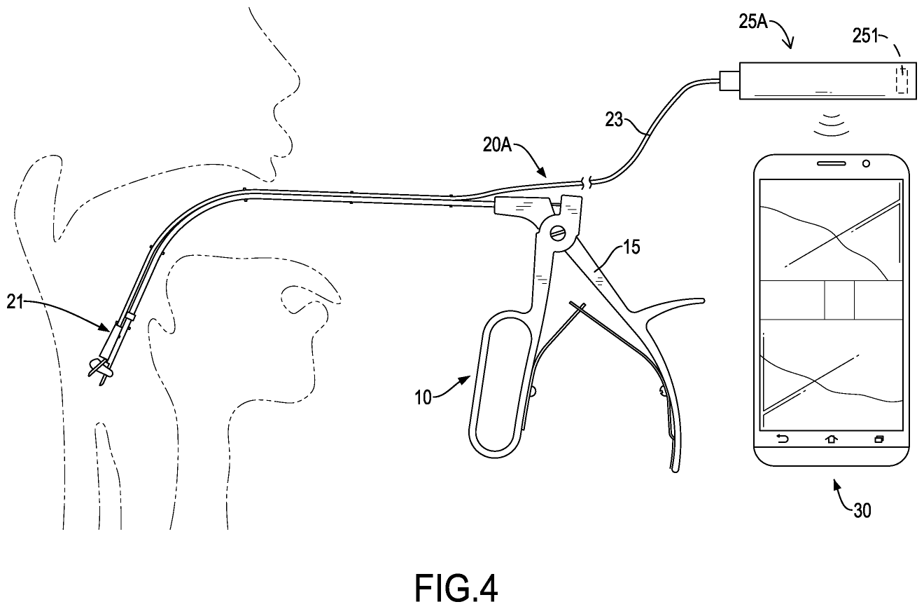

With reference to FIG. 4, in the second embodiment, the signal transmitter 25A is a wireless signal transmitter 25A connected to the tube body 23 and has a battery 251 mounted therein. The battery 251 can supply power to the image-guided device 20 and may be changeable or rechargeable. The wireless signal transmitter 25A can be connected to the external device 30 with wireless LAN or Bluetooth and can transmit signals to the external device 30 via radio waves. The image-guided device 20A is not connected to the external device 30 directly, and the image-guided pharyngolaryngeal forceps assembly may be used flexibly.

In another embodiment, the wireless signal transmitter 25A may include an electrical connector and a wireless signal transmitter unit connected to each other. The electrical connector is mounted on the tube body 23 and is detachably connected to the wireless signal transmitter unit. The signals can be transmitted to the external device 30 via radio waves when the electrical connector is connected to the wireless signal transmitter unit. The signals can be transmitted to the external device 30 via the electrical signal transmission connector, when the electrical connector is connected to the external device 30 directly.

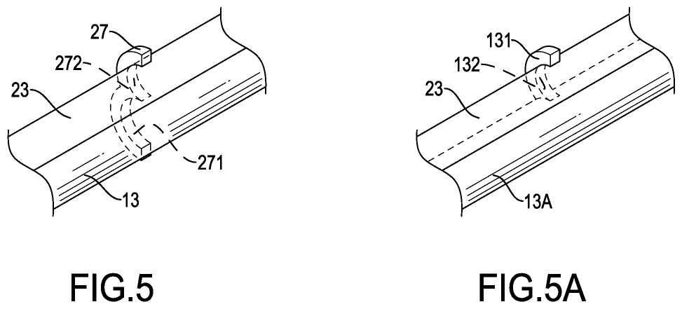

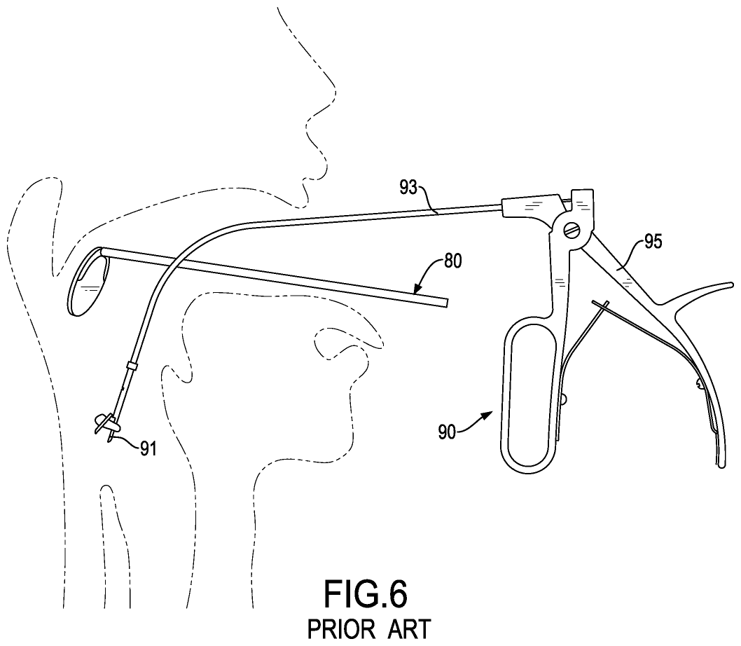

With reference to FIG. 5, the tube body 23 of the image-guided device 20 may be fixed on the body 13 of the pharyngolaryngeal forceps 10 with wire holders 27, such as clips, hooks, or straps, arranged along the body 13 at spaced intervals. The wire holder 27 may include various types. In this embodiment, the wire holder 27 has a W-shaped cross section with two C-shaped mounting cavities 271, 272. One of the mounting cavities 271 of the wire holder 27 is engaged with the body 13 of the pharyngolaryngeal forceps 10, and the other mounting cavity 272 of the wire holder 27 is engaged with the tube body 23 of the image-guided device 20.

With reference to FIG. 5A, the tube body 23 of the image-guided device 20 may be clamped in a holder portion 131 of the body 13 of the pharyngolaryngeal forceps 10. The holder portion 131 is formed in the body 13 and may have multiple clamping recesses 132 arranged along the body 13 at spaced intervals to clamp the image-guided device 20.

With reference to FIG. 3, when the first embodiment of the image-guided pharyngolaryngeal forceps assembly is in use, the signal transmitter 25 of the image-guided device 20, which is an electrical signal transmission connector, is connected to the external device 30. Power will be supplied from the external device 30 to the image-guided device 20 via the electrical connector. Consequently, the lens 211 of the camera module 21 can capture videos or images. The LEDs 213 can provide an illuminating effect. The image-guided device 20 will transmit signals to the external device 30 via the signal transmitter 25. The real time videos or images captured by the lens 211 can be displayed on the screen of the external device 30 after receiving and converting the signals from the image-guided device 20.

With reference to FIG. 4, when the second embodiment of the image-guided pharyngolaryngeal forceps assembly is in use, the image-guided device 20A is connected to the external device 30 via wireless LAN or Bluetooth, the battery 251 in the wireless signal transmitter 25A can supply the power to the image-guided device 20A, and the wireless signal transmitter 25A will transmit signals to the external device 30 via radio waves.

To perform a pharyngolaryngeal surgery, the surgeon will hold the image-guided pharyngolaryngeal forceps assembly with one hand, and hold the tongue of the patient with the other hand for unblocking the opening to the pharyngolaryngeal cavity of the patient. Then, the surgeon will insert the jaw assembly 11 of the pharyngolaryngeal forceps 10 into the pharyngolaryngeal cavity of the patient, the LEDs 213 of the camera module 21 will illuminate the pharynx and larynx of the patient, the lens 211 will capture the videos or images of the pharynx and larynx of the patient, and the real time videos or the images being captured by the camera module 21 will be displayed on the screen of the external device 30. The surgeon will be able to observe affected portion of the pharynx and larynx of the patient with enlarged images shown on the screen. The surgeon can move the pharyngolaryngeal forceps 10 to the affected portion under the real time videos or images shown on the screen to grasp and remove the diseased tissues with the jaw assembly 11. The surgeon can perform the pharyngolaryngeal surgery with two hands.

With such arrangements, the image-guided pharyngolaryngeal forceps assembly in accordance with the present invention can capture and transmit the real time videos or images to the external device 30 and can provide illumination during the pharyngolaryngeal surgery. The surgeon can hold the image-guided pharyngolaryngeal forceps assembly with one hand, hold the tongue of the patient to unblock the opening of laryngeal cavity with the other hand, and observe the pharynx and larynx of the patient displayed on the screen of the external device 30. The image-guided pharyngolaryngeal forceps assembly has advantages of simple structures, portability, flexibility, and mobility.

Compared to the conventional indirect pharyngolaryngoscopy surgery, with the image-guide pharyngolaryngeal forceps assembly of the present invention, the surgeon can perform the surgery without holding the indirect pharyngolaryngeal mirror and carrying the light source on the head during the surgery, and can perform the surgery by himself without assistance from the patient.

Compared to the conventional indirect pharyngolaryngoscopy surgery, by performing the pharyngolaryngeal surgery with the image-guide pharyngolaryngeal forceps assembly, the amount of instruments which are inserted in the pharyngolaryngeal cavity of the patient is decreased, traumas and infections during the surgery can be reduced, and the success rate of the surgery can be improved.

What is claimed is:

1. An image-guided pharyngolaryngeal forceps assembly comprising:
a pair of pharyngolaryngeal forceps having
a jaw assembly;
a body connected to the jaw assembly and being slender and curved; and
a handle connected to the jaw assembly via the body; and
an image-guided device fixed on the body of the pharyngolaryngeal forceps and having
a tube body mounted along the body of the pharyngolaryngeal forceps, being slender and flexible, and having
two ends; and
multiple electrical wires mounted in the tube body;
a camera module disposed on one of the ends of the tube body near the jaw assembly of the pharyngolaryngeal forceps and having a lens and multiple light-emitting diodes (LEDs) facing the jaw assembly of the pharyngolaryngeal forceps; and
a signal transmitter disposed on one of the ends of the flexible tube body near the handle of the pharyngolaryngeal forceps and connected to the camera module with the electrical wires of the tube body.

2. The image-guided pharyngolaryngeal forceps assembly as claimed in claim 1, wherein the signal transmitter of the image-guided device is an electrical connector.

3. The image-guided pharyngolaryngeal forceps assembly as claimed in claim 1, wherein the signal transmitter of the image-guided device is a wireless signal transmitter and has a battery mounted in the wireless signal transmitter.

4. The image-guided pharyngolaryngeal forceps assembly as claimed in claim 1, wherein the image-guided device is fixed on the body of the pharyngolaryngeal forceps with multiple wire holders.

5. The image-guided pharyngolaryngeal forceps assembly as claimed in claim 2, wherein the image-guided device is fixed on the body of the pharyngolaryngeal forceps with wire holders.

6. The image-guided pharyngolaryngeal forceps assembly as claimed in claim 3, wherein the image-guided device is fixed on the body of the pharyngolaryngeal forceps with wire holders.

7. The image-guided pharyngolaryngeal forceps assembly as claimed in claim 1, wherein the body of the pharyngolaryngeal forceps has a holder portion formed in the body of the pharyngolaryngeal forceps; and
the image-guided device is clamped in the holder portion of the body of the pharyngolaryngeal forceps.

8. The image-guided pharyngolaryngeal forceps assembly as claimed in claim 2, wherein the body of the pharyngolaryngeal forceps has a holder portion formed in the body of the pharyngolaryngeal forceps; and
the image-guided device is clamped in the holder portion of the body of the pharyngolaryngeal forceps.

9. The image-guided pharyngolaryngeal forceps assembly as claimed in claim 3, wherein the body of the pharyngolaryngeal forceps has a holder portion formed in the body of the pharyngolaryngeal forceps; and
the image-guided device is clamped in the holder portion of the body of the pharyngolaryngeal forceps.

10. The image-guided pharyngolaryngeal forceps assembly as claimed in claim 4, wherein
each wire holder has a W-shaped cross section to form two C-shaped mounting cavities; and
one of the two mounting cavities of each wire holder is engaged with the body of the pharyngolaryngeal forceps, and the other mounting cavity of the wire holder is engaged with the tube body of the image-guided device.

11. The image-guided pharyngolaryngeal forceps assembly as claimed in claim 5, wherein
each wire holder has a W-shaped cross section to form two C-shaped mounting cavities; and
one of the two mounting cavities of each wire holder is engaged with the body of the pharyngolaryngeal forceps, and the other mounting cavity of the wire holder is engaged with the tube body of the image-guided device.

12. The image-guided pharyngolaryngeal forceps assembly as claimed in claim 6, wherein
each wire holder has a W-shaped cross section to form two C-shaped mounting cavities; and
one of the two mounting cavities of each wire holder is engaged with the body of the pharyngolaryngeal forceps, and the other mounting cavity of the wire holder is engaged with the tube body of the image-guided device.
